(19) United States
(12) Patent Application Publication
(10) Pub. No.: US 2026/0167909 A1
(43) Pub. Date: Jun. 18, 2026

(54) SIGNAL-MEASURING DEVICE FOR CONTROLLING TEMPERATURE, CELL CULTURE APPARATUS COMPRISING THE SAME, AND MANUFACTURING METHOD OF SIGNAL MEASURING DEVICE

(71) Applicant: SAMSUNG ELECTRONICS CO., LTD., Suwon-si (KR)

(72) Inventor: Mikyung KIM, Suwon-si (KR)

(73) Assignee: SAMSUNG ELECTRONICS CO., LTD., Suwon-si (KR)

(21) Appl. No.: 19/263,006

(22) Filed: Jul. 8, 2025

(30) Foreign Application Priority Data
Dec. 18, 2024 (KR) 10-2024-0190106

(51) Int. Cl. C12M 1/38; C12M 1/34

(52) U.S. Cl. CPC C12M 1/38; C12M 41/12

(57) ABSTRACT

A method and a device are provided. A signal-measuring device may include: a plurality of measuring electrodes configured to contact a target object and measure a signal from the target object, wherein at least a portion of the plurality of measuring electrodes is outwardly exposed from the signal-measuring device in a first direction; a plurality of light sources configured to emit light in the first direction; and a heat transfer body on a path of the light emitted from at least one of the plurality of light sources, the heat transfer body configured to provide, to the target object, heat energy by the light emitted from the at least one of the plurality of light sources.

### CROSS-REFERENCE TO RELATED APPLICATION

This application claims priority from Korean Patent Application No. 10-2024-0190106, filed on Dec. 18, 2024, in the Korean Intellectual Property Office, the disclosure of which is incorporated herein by reference in its entirety.

### BACKGROUND

### 1. Field

Some embodiments of the present disclosure relate to a signal-measuring device for controlling a temperature, a cell culture apparatus including the same, and a manufacturing method of the signal-measuring device.

### 2. Description of Related Art

A signal-measuring device may include an electrode capable of contacting a target object and may collect electrical signals output from the target object via the electrode. For example, a signal-measuring device may sense a biosignal of a human or animal or a biosignal of a body organ (e.g., an organ or brain), an organoid, or a cell.

Said related art is information the inventor(s) acquired during the course of conceiving the present disclosure, or already possessed at the time, and is not necessarily prior art publicly known before the present application was filed.

### SUMMARY

One or more embodiments of the present disclosure may address at least the above problems and/or disadvantages not described above. Also, embodiments of the present disclosure are not required to overcome the disadvantages described above, and an embodiment of the present disclosure may not overcome any of the problems described above.

According to one or more embodiments of the present disclosure, a signal-measuring device may be provided and include: a plurality of measuring electrodes configured to contact a target object and measure a signal from the target object, wherein at least a portion of the plurality of measuring electrodes is outwardly exposed from the signal-measuring device in a first direction; a plurality of light sources configured to emit light in the first direction; and a heat transfer body on a path of the light emitted from at least one of the plurality of light sources, the heat transfer body configured to provide, to the target object, heat energy by the light emitted from the at least one of the plurality of light sources.

According to one or more embodiments of the present disclosure, the signal-measuring device may further include: a controller configured to control the plurality of light sources, wherein the controller is configured to independently turn at least some of the plurality of light sources on or off.

According to one or more embodiments of the present disclosure, the signal-measuring device may further include: a controller configured to control the plurality of light sources; and a temperature sensor configured to measure a temperature of the target object, wherein the controller is further configured to control an output of at least some of the plurality of light sources based on the temperature that is measured.

According to one or more embodiments of the present disclosure, the plurality of light sources includes: a first light source configured to emit a first light having a first wavelength; and a second light source configured to emit a second light having a second wavelength that is different from the first wavelength.

According to one or more embodiments of the present disclosure, the plurality of light sources includes: a first light source configured to provide heat stimulation, the first light source overlapping with the heat transfer body in the first direction; and a second light source configured to provide light stimulation, the second light source not overlapping with the heat transfer body in the first direction.

According to one or more embodiments of the present disclosure, the plurality of light sources includes: a first light source configured to provide heat stimulation, the first light source overlapping with the heat transfer body in the first direction; and a plurality of second light sources configured to provide light stimulation, the plurality of second light sources not overlapping with the heat transfer body in the first direction, wherein the plurality of second light sources includes: a second-first light source configured to emit first light having a first wavelength for the light stimulation; and a second-second light source configured to emit second light having a second wavelength for the light stimulation, the second wavelength being different from the first wavelength.

According to one or more embodiments of the present disclosure, at least one of the plurality of measuring electrodes includes a transparent electrode, wherein at least one of the plurality of light sources overlaps with the transparent electrode in the first direction.

According to one or more embodiments of the present disclosure, the heat transfer body includes a metal, conductive oxide, or graphene.

According to one or more embodiments of the present disclosure, the signal-measuring device may further include: a heat-blocking body configured to reduce heat emitted from the plurality of light sources or the heat transfer body from being transmitted to the target object, wherein the heat-blocking body includes a material having a heat transfer coefficient that is lower than a heat transfer coefficient of the heat transfer body.

According to one or more embodiments of the present disclosure, the heat-blocking body includes polyimide, an epoxy-based photoresist, or polydimethylsiloxane (PDMS).

According to one or more embodiments of the present disclosure, the signal-measuring device may further include: a transparent substrate, wherein the plurality of light sources are on a first side of the transparent substrate, wherein the plurality of measuring electrodes are on a second side of the transparent substrate, opposite to the first side in the first direction, and wherein at least a portion of the heat transfer body is on the second side of the transparent substrate.

According to one or more embodiments of the present disclosure, the plurality of measuring electrodes directly contacts a surface of the transparent substrate.

According to one or more embodiments of the present disclosure, the signal-measuring device may further include: an electrode protection layer including an insulating material, the electrode protection layer on the surface of the transparent substrate, wherein the electrode protection layer is configured to allow at least the portion of the plurality of measuring electrodes to be exposed outwardly toward the target object.

According to one or more embodiments of the present disclosure, the heat transfer body is on the electrode protection layer and is electrically insulated from an adjacent measuring electrode among the plurality of measuring electrodes.

According to one or more embodiments of the present disclosure, the transparent substrate includes: a substrate body including a transparent material, the substrate body being between the plurality of light sources and the plurality of measuring electrodes, the substrate body including a substrate via, wherein the substrate via overlaps with at least some of the plurality of light sources and the heat transfer body in a second direction that crosses the first direction.

According to one or more embodiments of the present disclosure, the heat transfer body contacts with a light source among the plurality of light sources, the light source overlapping with the heat transfer body in the first direction.

According to one or more embodiments of the present disclosure, the signal-measuring device may further include: a substrate, wherein the plurality of light sources and the plurality of measuring electrodes are on a same side of the substrate.

According to one or more embodiments of the present disclosure, the signal-measuring device may further include: a light source protection layer on the plurality of light sources such that the plurality of light sources are not outwardly exposed from the signal-measuring device, and an outer surface of the light source protection layer includes a flat shape; and an electrode protection layer on the outer surface of the light source protection layer and configured to allow at least the portion of the plurality of measuring electrodes to be outwardly exposed toward the target object, wherein the plurality of measuring electrodes directly contacts the outer surface of the light source protection layer.

According to one or more embodiments of the present disclosure, a cell culture apparatus may be provided and include a signal-measuring device configured to control a temperature, the signal-measuring device including: a plurality of measuring electrodes configured to contact a target object and measure a signal from the target object, wherein at least a portion of the plurality of measuring electrodes is outwardly exposed from the signal-measuring device in a first direction, a plurality of light sources configured to emit light in the first direction, and a heat transfer body on a path of the light emitted from at least some of the plurality of light sources, the heat transfer body configured to provide, to the target object, heat energy by the light emitted from the at least some of the plurality of light sources. The cell culture apparatus may further include: a container wall including a shape protruding from a surface of the signal-measuring device, wherein, in a view of the cell culture apparatus in the first direction, the shape surrounds the plurality of measuring electrodes, the plurality of light sources, and the heat transfer body.

According to one or more embodiments of the present disclosure, a manufacturing method of a signal-measuring device for controlling a temperature may be provided and include: providing a guide layer on a first surface of a substrate, the guide layer including a plurality of guide holes; inserting a plurality of light sources into the plurality of guide holes, respectively; providing a power line, the power line configured to supply power to the plurality of light sources; coating the plurality of light sources and the power line such that the plurality of light sources and the power line are not outwardly exposed; providing, on a second surface of the substrate, a plurality of measuring electrodes configured to contact a target object and measure a signal from the target object, wherein at least a portion of the plurality of measuring electrodes is exposed to the outside of the signal-measuring device; and providing a heat transfer body on a path along which at least some of the plurality of light sources are configured to emit light, wherein the at least some of the plurality of light sources are further configured to provide heat energy to the target object by emitting the light.

### BRIEF DESCRIPTION OF DRAWINGS

The above and/or other aspects will be more apparent by describing certain embodiments, taken in conjunction with the accompanying drawings, in which:

FIG. 1 is a perspective view illustrating a cell culture apparatus including a signal-measuring device, according to an embodiment;

FIG. 2 is a cross-sectional view illustrating a signal-measuring device according to an embodiment;

FIG. 3 is a block diagram illustrating a signal-measuring device according to an embodiment;

FIGS. 4 to 6 are diagrams illustrating an operation of a signal-measuring device, according to an embodiment;

FIG. 7 is a flowchart illustrating a control method of a signal-measuring device, according to an embodiment;

FIGS. 8A, 8B, 8C, 8D, 8E, and 8F are cross-sectional views illustrating a manufacturing method of a signal-measuring device, according to an embodiment;

FIG. 9 is a perspective view illustrating a cell culture apparatus including a signal-measuring device, according to an embodiment;

FIG. 10 is a cross-sectional view illustrating a signal-measuring device according to an embodiment;

FIG. 11 is a cross-sectional view illustrating a signal-measuring device according to an embodiment; and

FIG. 12 is a cross-sectional view illustrating a signal-measuring device according to an embodiment.

### DETAILED DESCRIPTION

The following detailed structural or functional description is provided as a non-limiting example only, and embodiments of the present disclosure may include various alterations and modifications. Embodiments of the present disclosure are not limited to example embodiments described herein, and should be understood to include all changes, equivalents, and replacements within the spirit and scope of the present disclosure.

Terms, such as “first,” “second,” and the like, may be used herein to describe components. Each of these terminologies is not used to define an essence, order, or sequence of a corresponding component, and are used merely to distinguish the corresponding component from other component(s). For example, a first component may be referred to as a second component, and similarly the second component may also be referred to as the first component.

It should be noted that if it is described that one component is “connected,” “coupled,” or “joined” to another component, a third component may be “connected,” “coupled,” and “joined” between the first and second components, although the first component may be directly connected, coupled, or joined to the second component.

As used herein, the singular forms “a,” “an,” and “the” include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components, and/or groups thereof.

As used herein, “A or B,” “at least one of A and B,” “at least one of A or B,” “A, B or C,” “at least one of A, B and C,” and “at least one of A, B, or C,” each of which may include any one of the items listed together in the corresponding one of the phrases, or all possible combinations thereof.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

Hereinafter, non-limiting example embodiments will be described in detail with reference to the accompanying drawings. When describing the example embodiments with reference to the accompanying drawings, like reference numerals refer to like elements and a repeated description related thereto may be omitted.

The same name may be used to describe an element included in the embodiments described herein and an element having a common function. Unless otherwise mentioned, descriptions of embodiments of the present disclosure may be applicable to other embodiments of the present disclosure, and thus, duplicated descriptions may be omitted for conciseness.

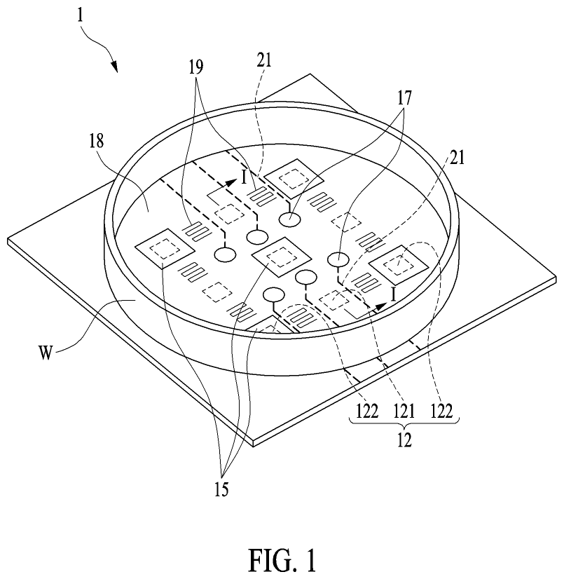

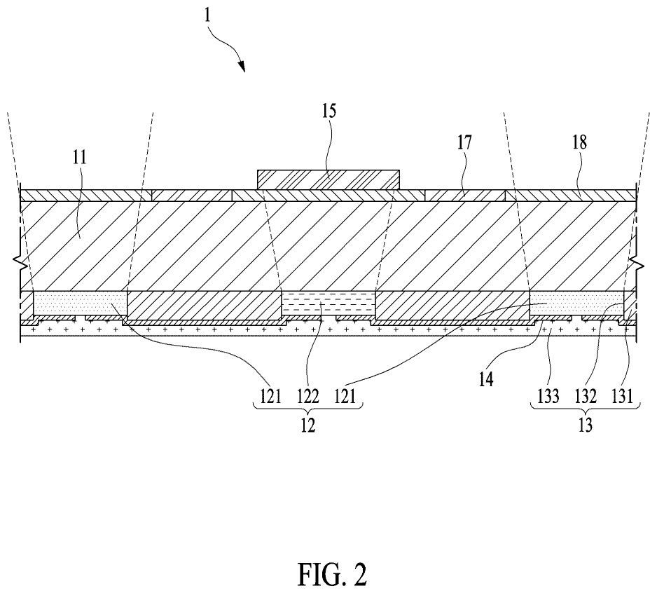

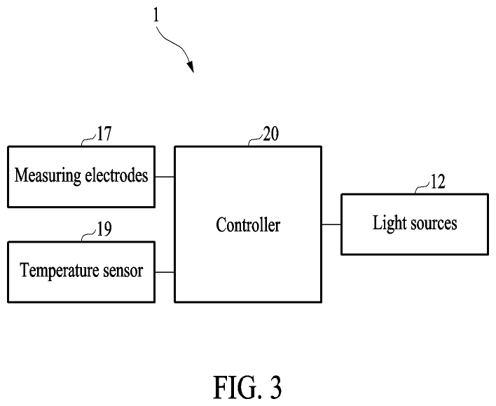

FIG. 1 is a perspective view illustrating a cell culture apparatus including a signal-measuring device, according to an embodiment. FIG. 2 is a cross-sectional view illustrating the signal-measuring device according to an embodiment. FIG. 3 is a block diagram illustrating a signal-measuring device according to an embodiment.

Referring to FIGS. 1 to 3, according to an embodiment, a signal-measuring device 1 may be applied to various systems that include biosignal measurement and temperature control. Although FIG. 1 illustrates an embodiment in which the signal-measuring device 1 is applied to the cell culture apparatus, the signal-measuring device 1 may also be applied to other fields, such as an implantable device, and a photothermal treatment technique and/or a drug delivery technique used in a cancer cell death method. For example, the cell culture apparatus may include the signal-measuring device 1 and a container wall W.

The container wall W may have a shape protruding from the surface of the signal-measuring device 1, and the shape may surround a plurality of measuring electrodes 17, a plurality of light sources 12, and a heat transfer body 15 when viewed in the protruding direction (e.g., the thickness direction of the signal-measuring device 1). Due to the container wall W, for example, a liquid target object may be prevented from leaking outwardly due to the shape that is covered by the container wall W and the signal-measuring device 1. In addition, while stably accommodating a target object, the temperature of the target object may be controlled or a response signal of the target object may be measured according to stimulation (e.g., light stimulation or heat stimulation) applied to the target object. The light stimulation and the heat stimulation may also be referred to as optical stimulation and thermal stimulation, respectively. The “target object” may include any material that may contact the outer surface of the signal-measuring device 1. For example, the target object may be in a liquid state or a solid state.

The signal-measuring device 1 may include a substrate 11, the light sources 12, a light source protection layer 13, a power line 14, the heat transfer body 15, the measuring electrodes 17, an electrode protection layer 18, a temperature sensor 19, and a controller 20. The signal-measuring device 1 having such a configuration may measure a biosignal output from the target object while controlling the temperature of the target object.

The substrate 11 may function as a base to form the light source protection layer 13. For example, the substrate 11 may be transparent. For example, the substrate 11 may be formed of glass. When the substrate 11 is transparent, the light sources 12 and the measuring electrodes 17 may be disposed on opposite sides of the substrate 11. For example, at least a portion of the heat transfer body 15 may be positioned on a side of the substrate 11 that is opposite to a side of the substrate 11 on which the light sources 12 are provided. With this structure, the light sources 12 and the measuring electrodes 17 may be disposed or formed by using each of two opposite surfaces of the substrate 11 manufactured to have high flatness so that the density of the measuring electrodes 17 may be improved by forming the measuring electrodes 17 with a fine scale (e.g., a micro-scale).

The light sources 12 may emit light in the direction (e.g., the up direction of FIG. 2) to which the measuring electrodes 17 are exposed in the signal-measuring device 1. For example, the light sources 12 may include a light-emitting diode (LED) (e.g., a micro-LED). For example, the light sources 12 may include a plurality of light sources that emits portions of light having different wavelengths. For example, the light sources 12 may include a first light source configured to emit light having a first wavelength, and a second light source configured to emit light having a second wavelength that is different from the first wavelength. With this configuration, a test may be performed while changing the wavelengths of light applied to the target object that exhibits different light responses depending on the wavelengths of light. For example, when the target object is a neuron cell, the light sources 12 may include a first light source configured to emit light having a blue wavelength to activate the neuron cell, and a second light source configured to emit light having a yellow wavelength to suppress the activity of the neuron cell.

According to an embodiment, the light sources 12 may include light sources 121 for light stimulation and light sources 122 for heat stimulation. That is, some of the light sources 12 may be used to provide light stimulation to the target object, and the remainder may be used to provide heat stimulation to the target object. Furthermore, some of the light sources 12 may be used to simultaneously provide light stimulation and heat stimulation to the target object. The light sources 12 may be referred to as the light sources 121 for light stimulation or the light sources 122 for heat stimulation. The light sources 121 and 122 may also be referred to as the optical stimulation light source 121 and the thermal stimulation light source 122, respectively. In some embodiments, the optical stimulation light source 121 and the thermal stimulation light source 122 may have the same hardware and operational structure but produce different intended effects, such as optically stimulating the target object, or thermally stimulating it, based on their respective arrangements within the signal-measuring device 1. In other embodiments, the optical stimulation light source 121 and the thermal stimulation light source 122 may have distinct structures and materials. For example, the optical stimulation light source 121 may include a plurality of different types of light sources to emit light at varying wavelengths (e.g., a blue light emitting diode (LED) to activate ion channels, and an ultraviolet (UV) LED to stimulate UV-sensitive proteins), whereas the thermal stimulation light source 122 may include a single type of light source (e.g., near-infrared (NIR) laser or light emitting diode (LED) that emits light in the range of approximately 800-1200 nm).

The light sources 121 for light stimulation may be disposed so as to not overlap with the heat transfer body 15 in the thickness direction (e.g., the vertical direction of FIG. 2) of the signal-measuring device 1. For example, the light sources 121 for light stimulation may be disposed in an area where transparent materials are stacked in the thickness direction of the signal-measuring device 1. For example, the light sources 121 for light stimulation may be disposed so as to not to overlap with the measuring electrodes 17 in the thickness direction of the signal-measuring device 1. For example, the light sources 121 for light stimulation may include various types of light sources configured to emit light having different wavelengths as described above. The light sources 122 for heat stimulation may be disposed so as to overlap with the heat transfer body 15 in the thickness direction of the signal-measuring device 1. With this configuration, the light stimulation and heat stimulation may be provided to the target object independently from each other

The light source protection layer 13 may cover the light sources 12 such that the light sources 12 are not outwardly exposed. The light source protection layer 13 may be formed of an insulating material, thereby protecting the light sources 12 and the power line 14 connected thereto. The light source protection layer 13 may include a guide layer 131, guide holes 132, and a coating layer 133.

The guide layer 131 may be formed of an insulating material. For example, a thickness (e.g., in the vertical direction of FIG. 2) of the guide layer 131 may be formed to be larger than a thickness of the light sources 12, thereby stably supporting the light sources 12. For example, the guide layer 131 may be formed in a shape that directly contacts one surface of the substrate 11. According to this shape, unintentional distortion of the light emission direction of the light sources 12 may be reduced by disposing the light sources 12 on one surface of the substrate 11 having relatively high flatness.

The guide holes 132 may be formed in a shape penetrating the guide layer 131. The guide holes 132 may be formed in a shape corresponding to the edge of the light sources 12, thereby accommodating the light sources 12. For example, a shape and size of the guide holes 132 may correspond to (e.g., be the same or substantially the same) as a shape and size of the light sources 12, respectively. Due to the guide holes 132, it may be possible to prevent a coating material of the light sources 12 from flowing and being exposed outwardly, in the process of coating the light sources 12 with the coating material.

The coating layer 133 may cover the light sources 12 and the power line 14 such that the light sources 12 and the power line 14 are not outwardly exposed. The coating layer 133 may be formed of, for example, the same insulating material as an insulating material of the guide layer 131, but is not limited thereto.

The power line 14 may be a conductive line that supplies power to the light sources 12 and may be formed, for example, for each of the light sources 12. With this configuration, power may individually be supplied, among the light sources 12, only to the light sources 12 positioned in a particular area, thereby providing localized light stimulation to the target object for each area. For example, the power line 14 may have a shape embedded between the guide layer 131 and the coating layer 133.

The heat transfer body 15 may be disposed on a path of light emitted from at least some of the light sources 12 (e.g., the light sources 122 for heat stimulation). The heat transfer body 15 may provide the target object with heat energy by the light emitted from at least some of the light sources 12 described above. For example, the heat transfer body 15 may be heated by heat emitted from at least some of the light sources 12 described above. For example, the heat transfer body 15 may be formed of a material having a higher heat transfer coefficient than a heat transfer coefficient of the substrate 11. For example, the heat transfer body 15 may be formed of a metal, conductive oxide (e.g., indium tin oxide (ITO)), and/or graphene. For example, the heat transfer body 15 may be formed of a color (e.g., black) having a high absorption rate for light. For example, the heat transfer body 15 may convert, into heat, at least a portion of the light emitted from at least some of the light sources 12 described above and may provide the converted heat to the target object. For example, the heat transfer body 15 may be formed of an opaque color (e.g., black) to increase absorption efficiency for light energy. For example, the heat transfer body 15 may include graphene oxide having high light-to-heat conversion efficiency. Such a material, which has higher light-to-heat conversion efficiency compared to other materials, may improve the energy efficiency of the signal-measuring device 1 and may help to quickly control the temperature.

According to an embodiment, compared to a case in which heat is generated from outside using a resistance-based heater and then supplied to the target object, heat may be more directly supplied to the target object by using the light sources 12 so that heat transfer efficiency may be improved. In addition, there may be the advantage of being able to perform two functions, light stimulation and temperature control by using the light sources 12 without the need for separate resistance.

For example, the heat transfer body 15 may have a cross-sectional width that is greater than the cross-sectional width of the light sources 122 for heat stimulation so that light emitted from the light sources 122 for heat stimulation is not directly transmitted to the target object. Furthermore, although FIGS. 1 and 2 illustrate that the heat transfer body 15 is disposed only in a partial area of the signal-measuring device 1, the heat transfer body 15 may be disposed over all areas of the signal-measuring device 1 except for the area where the measuring electrodes 17 are exposed in the signal-measuring device 1.

For example, the heat transfer body 15 may be disposed on the electrode protection layer 18. With this structure, the heat transfer body 15 may be disposed in a portion that directly contacts the target object, thereby reducing the waste of heat energy and improving heat transfer efficiency. For example, the heat transfer body 15 may be electrically insulated from the adjacent measuring electrodes 17 among the measuring electrodes 17 by the electrode protection layer 18.

At least a portion of the measuring electrodes 17 may be exposed to the outside of the signal-measuring device 1 and may measure a signal from the target object by contacting the target object. For example, the measuring electrodes 17 may be formed of microelectrodes. For ease of understanding, although FIGS. 1 and 2 illustrate a small number of measuring electrodes 17, the measuring electrodes 17 may be configured as a micro-electrode array (MEA) including a densely packed arrangement of micro-electrodes for precise measurement of each area of the target object. The micro-electrodes may be arranged in a grid or other configuration to record or simulate electrical activity at a high spatial resolution.

According to an embodiment, the measuring electrodes 17 and an electrode line 21, which may be connected thereto and transmit a signal outwardly, may be patterned in a form that directly contacts one surface (e.g., the upper surface in FIG. 2) of the substrate 11. For example, the surface of the substrate 11 may be formed of glass and may have high flatness. Using the flat surface of the substrate 11, the measuring electrodes 17 may be formed of high density, for example, in a scale of several tens of micros, thereby improving the measurement precision of the signal-measuring device 1.

According to an embodiment, at least a portion of the measuring electrodes 17 may be formed of a transparent electrode. For example, the measuring electrodes 17 may be formed of a transparent and conductive material (e.g., ITO). With this configuration, even when the light sources 12 (e.g., the light sources 121 for light stimulation) are disposed to overlap the measuring electrodes 17 in the thickness direction (e.g., the vertical direction of FIG. 2) of the signal-measuring device 1, the light stimulation may be provided to the target object through the measuring electrodes 17. Accordingly, since a signal from an area of the target object to which the light stimulation is applied may be directly measured, the accuracy and reliability of the measurement result of a response signal to the light stimulation may be improved.

The electrode protection layer 18 may allow at least a portion of the measuring electrodes 17 to be exposed outwardly toward the target object and allow an electrode line 21 connected to the measuring electrodes 17 not to be exposed outwardly, and may cover the measuring electrodes 17 and/or the electrode line 21. The electrode protection layer 18 may be formed of an insulating material and may protect the measuring electrodes 17 and the electrode line 21 connected thereto.

The temperature sensor 19 may measure the temperature of the target object. For example, the temperature sensor 19 may be formed adjacent to the heat transfer body 15 and may identify whether heat transmitted through the heat transfer body 15 is normally controlled. For example, the temperature sensor 19 may be stacked on the heat transfer body 15. For example, the temperature sensor 19 may be formed of a thin metal line formed in a zigzag shape, and the controller 20 may detect a change in temperature based on a change in resistance of the temperature sensor 19.

The controller 20 may control the light sources 12. For example, the controller 20 may independently turn at least some of the light sources 12 on or off. Through this configuration, the signal-measuring device 1 may locally transmit heat to a partial area of the target object. Accordingly, the signal-measuring device 1 may identify the responses to different levels of heat stimulation for each area. In addition, when it is not needed to identify a heat stimulation response over all areas of the target object, the energy efficiency of the signal-measuring device 1 may be improved by only locally providing the heat stimulation to the target object. For example, the controller 20 may control the degree of heat transfer to the target object by controlling the intensity and/or the on-time of at least some of the light sources 12.

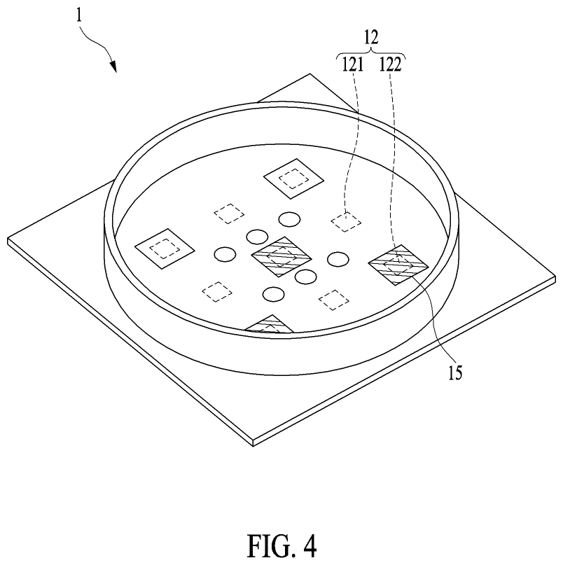

FIGS. 4 to 6 are diagrams illustrating an operation of a signal-measuring device, according to an embodiment.

Referring to FIGS. 3 and 4, the controller 20 may control the temperature of a target object by, for example, turning on only the light sources 122 for heat stimulation among the light sources 12. In this case, the response to light stimulation may be excluded and only the response to heat stimulation may be measured by turning off the light sources 121 for light stimulation among the light sources 12. For example, as shown in FIG. 4, the controller 20 may locally provide heat to the target object by turning on only some of the light sources 122 for heat stimulation positioned in a particular area.

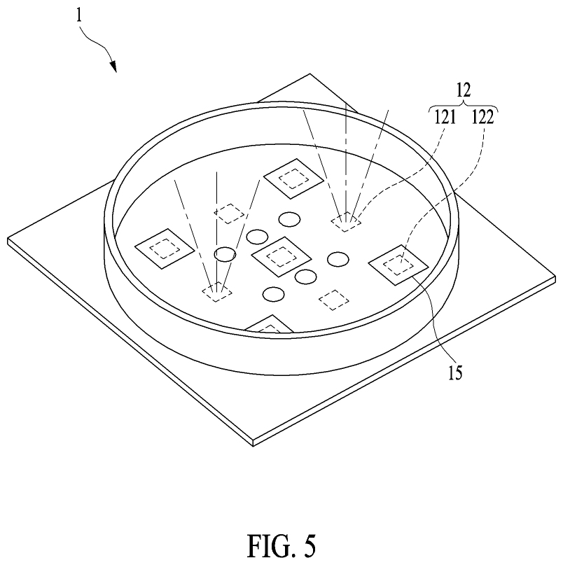

Referring to FIGS. 3 and 5, the controller 20 may provide light stimulation to the target object by, for example, turning on only the light sources 121 for light stimulation among the light sources 12. In this case, the response to heat stimulation may be excluded and only the response to light stimulation may be measured by turning off the light sources 122 for heat stimulation among the light sources 12. For example, as shown in FIG. 5, the controller 20 may locally provide light to the target object by turning on only some of the light sources 121 for light stimulation positioned in a particular area. For example, the controller 20 may turn on only some of the light sources 121 for light stimulation that emit light of a set wavelength.

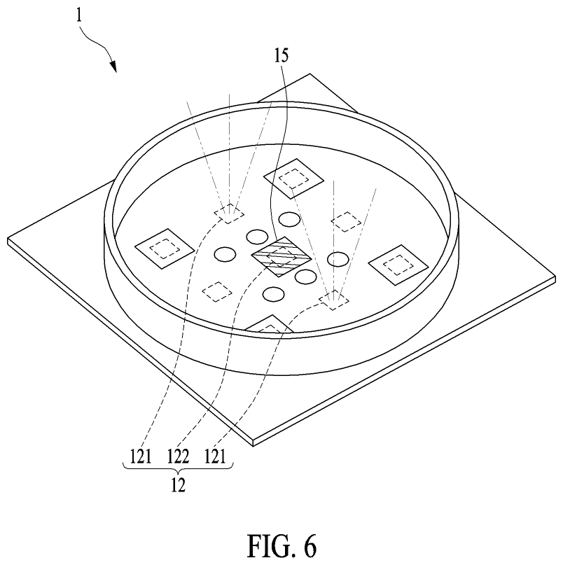

Referring to FIGS. 3 and 6, the controller 20 may simultaneously turn on the light sources 121 for light stimulation and the light sources 122 for heat stimulation. Through this configuration, a user may collect response signals to light stimulation at the set temperature.

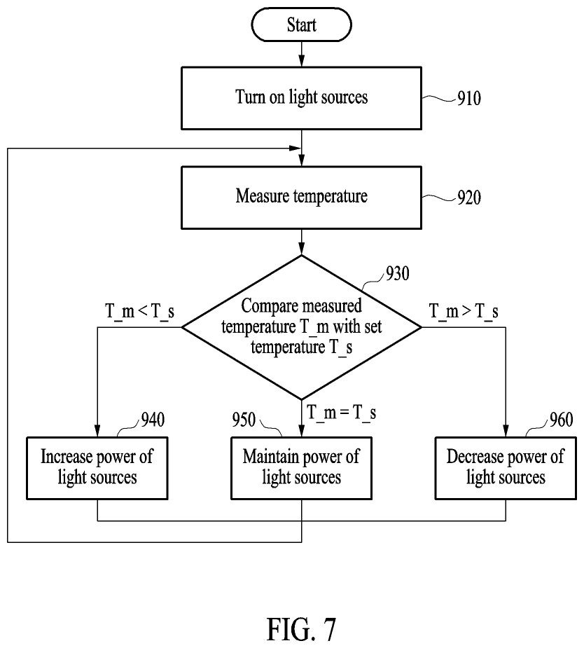

FIG. 7 is a flowchart illustrating a control method a signal-measuring device, according to an embodiment.

Referring to FIGS. 3 and 7, according to an embodiment, the controller 20 of the signal-measuring device 1 may control an output of at least some of the light sources 12 based on the temperature sensed by the temperature sensor 19. According to an embodiment, the control method of the signal-measuring device 1 may include an operation 910 of turning on the light sources 12, an operation 920 of measuring the temperature, an operation 930 of comparing a measured temperature T_m with a set temperature T_s, an operation 940 of increasing power of the light sources 12, an operation 950 of maintaining power of the light sources 12, and an operation 960 of decreasing power of the light sources 12. Unless otherwise stated, the order of the above-described operations is not limited and may be performed reversely or simultaneously. In addition, some of the operations may be omitted.

In the operation 910, at least some of the light sources 12 may be turned on. In the operation 920, the temperature of a target object may be measured using the temperature sensor 19. In the operation 930, the controller 20 may compare the measured temperature T_m received from the temperature sensor 19 with the set temperature T_s. Here, the set temperature T_s may be, for example, a temperature that is input from a user or an external terminal or a temperature according to a set condition stored in the controller 20.

When the measured temperature T_m is lower than the set temperature T_s according to the comparison result in the operation 930, the controller 20 may increase the power of the light sources 12 in the operation 940. When the measured temperature T_m is the same as the set temperature T_s in the operation 930, the controller 20 may maintain the power of the light sources 12 in the operation 950. When the measured temperature T_m is higher than the set temperature T_s in the operation 930, the controller 20 may decrease the power of the light sources 12 in the operation 960. Furthermore, the set temperature T_s may not only be a temperature having a certain value but also a temperature range having a certain range. Through such a feedback temperature maintenance function, the temperature of the target object may be maintained at the temperature for signal measurement.

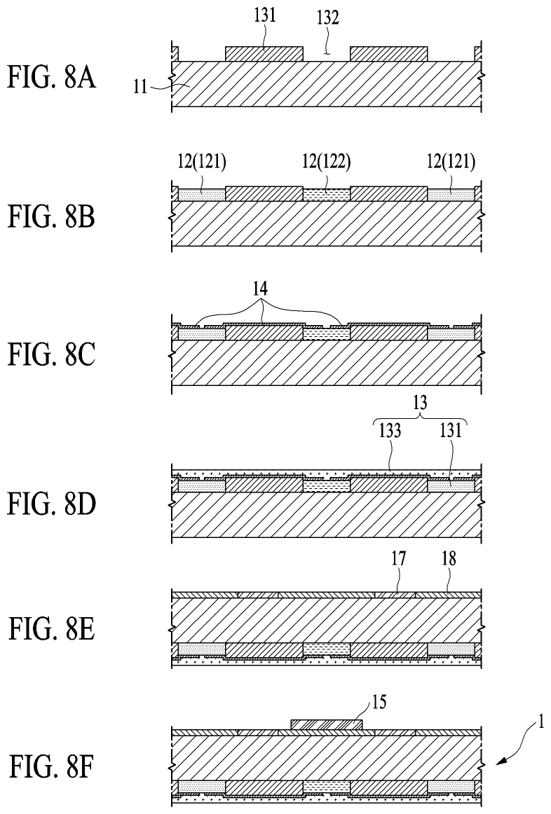

FIGS. 8A-8F are cross-sectional views illustrating a manufacturing method of a signal-measuring device, according to an embodiment

As shown in FIG. 8A, the guide layer 131, in which the guide holes 132 are formed, may be formed on one surface of the substrate 11 (e.g., the upper surface in FIG. 8A). For example, the guide layer 131 may be formed through a photolithography process using a photoresist. For example, the height of the guide layer 131 may be higher than a height of the light sources 12 to be inserted into the guide holes 132. With this configuration, the problem of departure of the light sources 12, which may occur due to the movement or motion of a target object, may be reduced. Furthermore, unless otherwise stated, the scope of the present disclosure also includes a case in which the height of the guide layer 131 is lower than or equal to the height of the light sources 12. As shown in FIG. 8B, when the light sources 12 are inserted into the guide holes 132, respectively, the power line 14 that supplies power to the light sources 12 may be formed, as shown in FIG. 8C. For example, the power line 14 may be formed using a plating process and a photolithography process.

As shown in FIG. 8D, the light sources 12 and the power line 14 may be coated with a coating material such as to not to be outwardly exposed. The coating layer 133 and the guide layer 131 formed by the coating material may be collectively referred to as the light source protection layer 13. Furthermore, due to the height difference between the guide layer 131 and the light sources 12, there may be a possibility that a portion of the coating layer 133 corresponding to the guide holes 132 has a recessed shape. When the outer surface of the coating layer 133 is not formed flat, it may be difficult to perform a precise patterning process on the outer surface of the coating layer 133.

As shown in FIG. 8E, when the measuring electrodes 17 are formed on the other surface of the substrate 11 (e.g., the lower surface in FIG. 8D, which is the upper surface in FIG. 8E), the measuring electrodes 17 (e.g., an electrode pad and an electrode line connected thereto) having a finer scale and a more precise shape than the outer surface of the coating layer 133 may be formed. For example, the measuring electrodes 17 may be formed using a plating process and a photolithography process. At least a portion (e.g., an electrode pad) of the measuring electrodes 17 may be exposed to the outside of the signal-measuring device 1 and may measure a signal from the target object by contacting the target object. The remaining portion (e.g., an electrode line) of the measuring electrodes 17 may be covered by the electrode protection layer 18 such as to not to be outwardly exposed. The electrode protection layer 18 may be formed of an insulating material such as, for example, an epoxy-based photoresist (e.g., SU-8), polyimide, parylene C oxide, and/or nitride. For example, the electrode protection layer 18 may be formed through a photolithography process using a photoresist.

According to an embodiment, as shown in the order of FIGS. 8A-8F, after the light sources 12 are disposed on one surface of the substrate 11, the measuring electrodes 17 may be formed on the other surface of the substrate 11. According to this order, with the light sources 12 disposed first, the measuring electrodes 17 may be formed with a relatively fine scale compared to the light sources 12 and the power line 14 according to the manufacturing error of the light sources 12 so that the yield of the good product of the signal-measuring device 1 may increase.

As shown in FIG. 8F, the heat transfer body 15 may be formed to define at least a portion of the outer surface of the signal-measuring device 1. According to an embodiment, a temperature sensor (e.g., the temperature sensor 19 in FIG. 1) may be formed adjacent to or on the heat transfer body 15. For example, when the heat transfer body 15 and the temperature sensor 19 are formed of the same metal material as each other, the temperature sensor 19 and the heat transfer body 15 may be formed using the same metal wiring process.

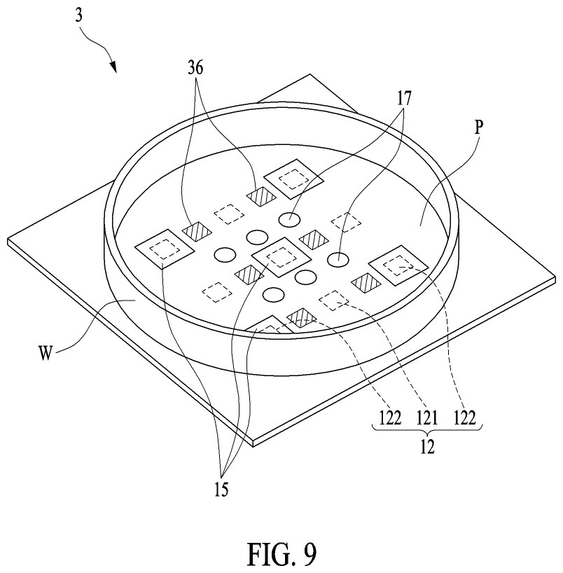

Hereinafter, a component, which has the same common function as a component included in any one embodiment of the present disclosure, is described by using the same name for other embodiments of the present disclosure. Unless otherwise stated, the configuration of any one embodiment of the present disclosure may be applied to other embodiments of the present disclosure, and the specific description of the repeated configuration may be omitted. For example, a heat-blocking body 36 illustrated in FIG. 9 may be applied to the embodiments shown in FIG. 1 or FIGS. 10 to 12.

FIG. 9 is a perspective view illustrating a cell culture apparatus including a signal-measuring device, according to an embodiment.

Referring to FIG. 9, according to an embodiment, a signal-measuring device 3 may include a device plate P, the light sources 12, the heat transfer body 15, the measuring electrodes 17, and the heat-blocking body 36. The device plate P may include a substrate (e.g., the substrate 11 of FIG. 2), a light source protection layer (e.g., the light source protection layer 13 of FIG. 2), and an electrode protection layer (e.g., the electrode protection layer 18 of FIG. 2). The light sources 12 may include the light sources 121 for light stimulation and the light sources 122 for heat stimulation.

According to an embodiment, the heat-blocking body 36 may reduce heat emitted from the light sources 12 or the heat transfer body 15 from being transmitted to a target object. The heat-blocking body 36 may be formed of a material having a lower heat transfer coefficient than a heat transfer coefficient of the heat transfer body 15, thereby locally lowering the temperature of the target object that is adjacent to an area where the heat-blocking body 36 is positioned. For example, the heat-blocking body 36 may be formed of a material having a lower heat transfer coefficient than a heat transfer coefficient of the substrate 11. For example, the heat-blocking body 36 may include polyimide, an epoxy-based photoresist (e.g., SU-8), or polydimethylsiloxane (PDMS). When the signal-measuring device 3 is used in a drug delivery process, this configuration may help maintain the stability of the drug or protect the tissue.

The heat-blocking body 36 may be disposed on, for example, the electrode protection layer 18. With this structure, the heat-blocking body 36 may be disposed in a portion that directly contacts the target object, thereby improving the temperature control efficiency of the target object.

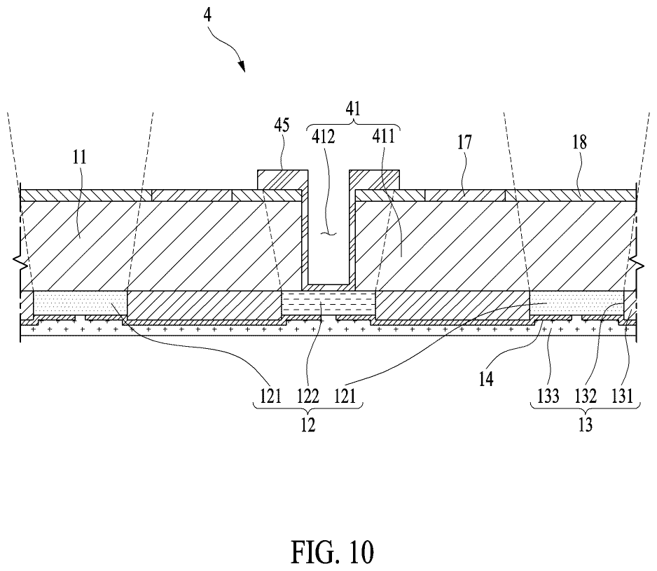

FIG. 10 is a cross-sectional view illustrating a signal-measuring device according to an embodiment.

Referring to FIG. 10, according to an embodiment, a signal-measuring device 4 may include a substrate 41, the light sources 12, the light source protection layer 13, the power line 14, a heat transfer body 45, the measuring electrodes 17, and the electrode protection layer 18. The light sources 12 may include the light sources 121 for light stimulation and the light sources 122 for heat stimulation. The light source protection layer 13 may include the guide layer 131, the guide holes 132, and the coating layer 133.

According to an embodiment, the substrate 41 may include a substrate body 411 and a substrate via 412. The substrate body 411 may be positioned between the light sources 12 and the measuring electrodes 17 and may be formed of a transparent material (e.g., glass).

The substrate via 412 may be formed by penetrating the substrate body 411. The substrate via 412 may overlap at least some of the light sources 12 (e.g., the light sources 122 for heat stimulation) and the heat transfer body 45 based on the thickness direction (e.g., the vertical direction of FIG. 10) of the signal-measuring device 4. With this configuration, energy loss generated in the process in which heat, which is transmitted from the light sources 12 toward the target object, passes through the substrate 41 may be reduced.

For example, at least a portion of the heat transfer body 45 may be formed to be in contact with, among the light sources 12, the light sources 12 (e.g., the light sources 122 for heat stimulation) that overlap with the heat transfer body 45 based on the thickness direction of the signal-measuring device 4. With this shape, faster and more accurate temperature control may be performed in addition to the improvement of energy efficiency by improving the heat transfer efficiency from the light sources 12 to the target object.

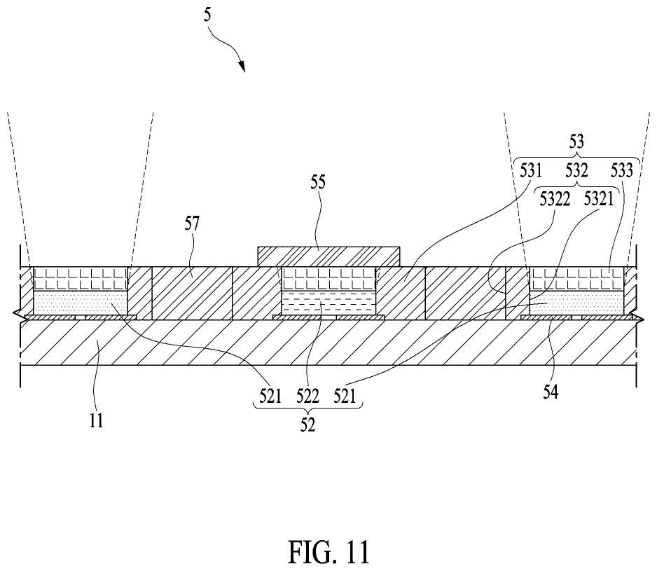

FIG. 11 is a cross-sectional view illustrating a signal-measuring device according to an embodiment.

Referring to FIG. 11, according to an embodiment, a signal-measuring device 5 may include the substrate 11, light sources 52, a protection layer 53, a power line 54, a heat transfer body 55, and measuring electrodes 57. The light sources 52 may include light sources 521 for light stimulation and light sources 522 for heat stimulation.

According to an embodiment, the light sources 52 and the measuring electrodes 57 may be disposed in the same direction with respect to the substrate 11. For example, the light sources 52 and the measuring electrodes 57 may be installed in substantially the same layer. For example, the light sources 52 and the measuring electrodes 57 may overlap with each other in a horizontal direction (e.g., a horizontal direction of FIG. 11). With this structure, the thickness of the signal-measuring device 5 may be reduced.

The protection layer 53 may perform the functions of a light source protection layer (e.g., the light source protection layer 13 of FIG. 2) and an electrode protection layer (e.g., the electrode protection layer 18 of FIG. 2) according to the embodiments described above. Unless otherwise stated, the description of the light source protection layer 13 and the electrode protection layer 18 may be applied to the protection layer 53. The protection layer 53 may include a guide layer 531, guide holes 532, and a coating layer 533.

The guide holes 532 may include a light source guide hole 5321 and an electrode guide hole 5322. The light sources 52 and the measuring electrodes 57 may be inserted into the light source guide hole 5321 and the electrode guide hole 5322, respectively. The coating layer 533 may cover the light sources 52 so that the light sources 52 are not directly exposed outwardly.

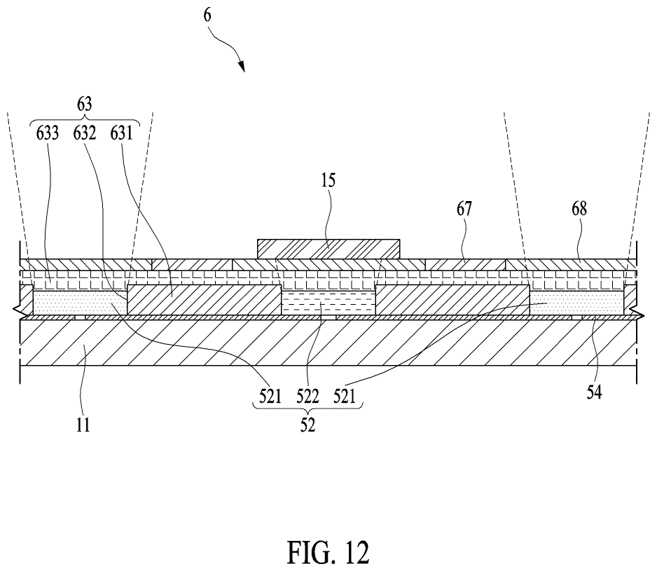

FIG. 12 is a cross-sectional view illustrating a signal-measuring device according to an embodiment.

Referring to FIG. 12, according to an embodiment, a signal-measuring device 6 may include the substrate 11, the light sources 52, a light source protection layer 63, the power line 54, the heat transfer body 15, measuring electrodes 67, and an electrode protection layer 68. The light sources 52 may include the light sources 521 for light stimulation and the light sources 522 for heat stimulation. The light source protection layer 63 may include a guide layer 631, guide holes 632, and a coating layer 633.

The coating layer 633 may cover the light sources 52 so that the light sources 52 are not exposed outwardly, and may have a flat outer surface. For example, to prevent formation of a portion of the coating layer 633 from being recessed into the guide holes 632, the coating layer 633 may be formed with a sufficient thickness or the coating layer 633 may be polished after the coating layer 633 is formed so that the outer surface of the coating layer 633 may have a flat shape. For example, the coating layer 633 may include a first coating layer to cover the light sources 52 and a second coating layer to flatten the outer surface of the coating layer 633 by being stacked on the first coating layer. The first coating layer and the second coating layer may be formed of different materials from each other.

The measuring electrodes 67 and an electrode line connected thereto may be patterned in a form that directly contacts the outer surface of the light source protection layer 63, that is, the flat outer surface of the coating layer 633. According to this structure, compared to the embodiment described with reference to FIG. 11, the density of the measuring electrodes 67 may be improved by forming the measuring electrodes 67 with a fine scale (e.g., a micro-scale).

The electrode protection layer 68 may be formed on the outer surface of the light source protection layer 63. The electrode protection layer 68 may allow at least a portion of the measuring electrodes 67 to be exposed outwardly toward the target object and may cover the electrode line, which is connected to the measuring electrodes 67 and transmits a signal outwardly, not to be exposed outwardly.

As described above, although non-limiting example embodiments of the present disclosure have been described with reference to the drawings, a person skilled in the art may apply various technical modifications and variations based thereon. For example, suitable results may be achieved if the described techniques are performed in a different order and/or if components in a described system, architecture, device, or circuit are combined in a different manner and/or replaced or supplemented by other components or their equivalents. Therefore, such modifications and variations, and other implementations, other embodiments, and equivalents are included within the scope of present disclosure

What is claimed is:

1. A signal-measuring device comprising:
a plurality of measuring electrodes configured to contact a target object and measure a signal from the target object, wherein at least a portion of the plurality of measuring electrodes is outwardly exposed from the signal-measuring device in a first direction;
a plurality of light sources configured to emit light in the first direction; and
a heat transfer body on a path of the light emitted from at least one of the plurality of light sources, the heat transfer body configured to provide, to the target object, heat energy by the light emitted from the at least one of the plurality of light sources.

2. The signal-measuring device of claim 1, further comprising:
a controller configured to control the plurality of light sources,
wherein the controller is configured to independently turn at least some of the plurality of light sources on or off.

3. The signal-measuring device of claim 1, further comprising:
a controller configured to control the plurality of light sources; and
a temperature sensor configured to measure a temperature of the target object,
wherein the controller is further configured to control an output of at least some of the plurality of light sources based on the temperature that is measured.

4. The signal-measuring device of claim 1, wherein the plurality of light sources comprises:
a first light source configured to emit a first light having a first wavelength; and
a second light source configured to emit a second light having a second wavelength that is different from the first wavelength.

5. The signal-measuring device of claim 1, wherein the plurality of light sources comprises:
a first light source configured to provide heat stimulation, the first light source overlapping with the heat transfer body in the first direction; and
a second light source configured to provide light stimulation, the second light source not overlapping with the heat transfer body in the first direction.

6. The signal-measuring device of claim 1, wherein the plurality of light sources comprises:
a first light source configured to provide heat stimulation, the first light source overlapping with the heat transfer body in the first direction; and
a plurality of second light sources configured to provide light stimulation, the plurality of second light sources not overlapping with the heat transfer body in the first direction,
wherein the plurality of second light sources comprises:
a second-first light source configured to emit first light having a first wavelength for the light stimulation; and
a second-second light source configured to emit second light having a second wavelength for the light stimulation, the second wavelength being different from the first wavelength.

7. The signal-measuring device of claim 1, wherein at least one of the plurality of measuring electrodes comprises a transparent electrode,
wherein at least one of the plurality of light sources overlaps with the transparent electrode in the first direction.

8. The signal-measuring device of claim 1, wherein the heat transfer body comprises a metal, conductive oxide, or graphene.

9. The signal-measuring device of claim 1, further comprising:
a heat-blocking body configured to reduce heat emitted from the plurality of light sources or the heat transfer body from being transmitted to the target object,
wherein the heat-blocking body comprises a material having a heat transfer coefficient that is lower than a heat transfer coefficient of the heat transfer body.

10. The signal-measuring device of claim 9, wherein the heat-blocking body comprises polyimide, an epoxy-based photoresist, or polydimethylsiloxane (PDMS).

11. The signal-measuring device of claim 1, further comprising:
a transparent substrate,
wherein the plurality of light sources are on a first side of the transparent substrate,
wherein the plurality of measuring electrodes are on a second side of the transparent substrate, opposite to the first side in the first direction, and
wherein at least a portion of the heat transfer body is on the second side of the transparent substrate.

12. The signal-measuring device of claim 11, wherein the plurality of measuring electrodes is configured to directly contact a surface of the transparent substrate.

13. The signal-measuring device of claim 12, further comprising:
an electrode protection layer comprising an insulating material, the electrode protection layer on the surface of the transparent substrate,
wherein the electrode protection layer is configured to allow at least the portion of the plurality of measuring electrodes to be exposed outwardly toward the target object.

14. The signal-measuring device of claim 13, wherein the heat transfer body is on the electrode protection layer and is electrically insulated from an adjacent measuring electrode among the plurality of measuring electrodes.

15. The signal-measuring device of claim 11, wherein the transparent substrate comprises:
a substrate body comprising a transparent material, the substrate body being between the plurality of light sources and the plurality of measuring electrodes, the substrate body including a substrate via,
wherein the substrate via overlaps with at least some of the plurality of light sources and the heat transfer body in a second direction that crosses the first direction.

16. The signal-measuring device of claim 15, wherein the heat transfer body contacts with a light source among the plurality of light sources, the light source overlapping with the heat transfer body in the first direction.

17. The signal-measuring device of claim 1, further comprising:
a substrate,
wherein the plurality of light sources and the plurality of measuring electrodes are on a same side of the substrate.

18. The signal-measuring device of claim 17, further comprising:
a light source protection layer on the plurality of light sources such that the plurality of light sources are not outwardly exposed from the signal-measuring device, and an outer surface of the light source protection layer comprises a flat shape; and
an electrode protection layer on the outer surface of the light source protection layer and configured to allow at least the portion of the plurality of measuring electrodes to be outwardly exposed toward the target object,
wherein the plurality of measuring electrodes directly contacts the outer surface of the light source protection layer.

19. A cell culture apparatus comprising:
a signal-measuring device configured to control a temperature, the signal-measuring device comprising:
a plurality of measuring electrodes configured to contact a target object and measure a signal from the target object, wherein at least a portion of the plurality of measuring electrodes is outwardly exposed from the signal-measuring device in a first direction,
a plurality of light sources configured to emit light in the first direction; and
a heat transfer body on a path of the light emitted from at least some of the plurality of light sources, the heat transfer body configured to provide, to the target object, heat energy by the light emitted from the at least some of the plurality of light sources; and
a container wall comprising a shape protruding from a surface of the signal-measuring device, wherein, in a view of the cell culture apparatus in the first direction, the shape surrounds the plurality of measuring electrodes, the plurality of light sources, and the heat transfer body.

20. A manufacturing method of a signal-measuring device for controlling a temperature, the manufacturing method comprising:
providing a guide layer on a first surface of a substrate, the guide layer including a plurality of guide holes;
inserting a plurality of light sources into the plurality of guide holes, respectively;
providing a power line, the power line configured to supply power to the plurality of light sources;
coating the plurality of light sources and the power line such that the plurality of light sources and the power line are not outwardly exposed;
providing, on a second surface of the substrate, a plurality of measuring electrodes configured to contact a target object and measure a signal from the target object, wherein at least a portion of the plurality of measuring electrodes is exposed to the outside of the signal-measuring device; and
providing a heat transfer body on a path along which at least some of the plurality of light sources are configured to emit light,
wherein the at least some of the plurality of light sources are further configured to provide heat energy to the target object by emitting the light.
